(19) United States
(12) Patent Application Publication
(10) Pub. No.: US 2017/0172487 A1
(43) Pub. Date: Jun. 22, 2017

(54) IMAGING SKIN CANCER DETECTION DEVICE

(71) Applicant: Oren Aharon, Haifa (IL)

(72) Inventor: Oren Aharon, Haifa (IL)

(21) Appl. No.: 15/156,359

(22) Filed: May 17, 2016

(30) Foreign Application Priority Data
Dec. 21, 2015 (IL) 243259

(51) Int. Cl. A61B 5/00

(52) U.S. Cl. CPC A61B 5/444; A61B 5/0077; A61B 5/6898; A61B 5/70; A61B 5/0075; A61B 2560/0431; A61B 2562/0238; A61B 2562/04; A61B 2576/02

(57) ABSTRACT

The current invention relates to medical analysis of skin Basal-cell carcinoma and melanoma by sub skin infrared imaging and 3-D tomographic reconstruction of malignant images.

It is the purpose of this invention to provide methods and compositions to reliably collect data of malignant and benign lesions on the skin and beneath the surface of the skin in a non-invasive manner.

Detection of a skin cancer is carried out by physical examination of the skin, or by imaging technologies of the epidermis. This is followed by biopsy of selected lesions suspected to be cancerous.

The diagnostic methods of the invention are based on external imaging of lesions combined with an image taken with sub skin illumination revealing under the skin structures. A combination of the two images increases the available data, offering a better diagnostic tool.

### BACKGROUND OF THE INVENTION

1. Field of Invention

The current invention relates to medical analysis of skin Basal-cell carcinoma and melanoma by sub skin infrared imaging and 3-D tomographic reconstruction of malignant images.

2. Description of the Related Art

Melanoma is a type of cancer that develops from melanocytes. Melanoma has a tendency to spread; it develops on the surface of the skin and later spreads under the skin, making it essential to diagnose and treat. It is the most dangerous type of skin cancer, and its prevalence is increasing at an alarming rate. Globally, 1 in 50 men and women will be diagnosed with melanoma of the skin during their lifetime.

Basal Cell Carcinoma (BCC) is a condition where abnormal, uncontrolled growths or lesions arise in the skin's basal cells, which line the deepest layer of the epidermis.

Nowadays, the diagnosis for these diseases relies mainly on the expertise of the dermapathologist and chances of survival increase greatly if the detection is given at an early stage.

More than one out of every three new cancers is a skin cancer, and the vast majority are BCCs. Combined with the fact that both BCC and Melanoma are usually curable in their early stages, these statistics prove the importance of finding new ways to detect skin cancers at an early stage so that patients can receive the most favorable treatment. Since Melanoma also may begin in or near a mole, it is also important to monitor the appearance of moles and other dark spots on the skin. Generally the term “skin lesion” is used to describe areas which are suspected to be a tumor, and usually the detection methods include taking samples from such areas.

One method for detection could be obtained by using Infrared imaging- recent modifications in infrared technology have increased interest for infrared imaging in the field of medicine. By using the advantages of these modifications, it is possible to perform check ups on patients in such way that would be both non-invasive and efficient in detecting abnormalities in the skin, and with the current invention it will be possible to do so not only in the Epidermis, but also in the Dermis and Subcutaneous layers, and thus receive more data from each skin sample.

### SUMMARY OF THE INVENTION

It is the purpose of this invention to provide methods and compositions to reliably collect data of malignant and benign lesions on the skin and beneath the surface of the skin in a non-invasive manner.

Detection of a skin cancer is carried out by physical examination of the skin, or by imaging technologies of the epidermis. This is followed by biopsy of selected lesions suspected to be cancerous.

The effectiveness of this procedure is dependent on the experience of the examiner or the quality and interpretation of image or biopsy, sometimes non diagnosed cancer and errors can be fatal. Today, due to diagnosis complexity and lack of information there is no reliable way for automatic cancer detection. Occasionally, undetected cancers will metastasize beyond the original site of occurrence and lead to major complications. On the other hand, many unnecessary biopsies occur with high frequency. On some instances the biopsy is performed on the facial region and may result in some incidents of cosmetic scarring.

The diagnostic methods of the invention are based on external imaging of lesions combined with an image taken with sub skin illumination revealing under the skin structures. A combination of the two images increases the available data, offering a better diagnostic tool. Moreover, the method of illumination will also offer a way of probing the area for local blood oximetry. The tool is useful for home-use, by a non-professional individual for performing a prolonged tumor growth follow-up or for the use in the clinic by a health professional individual. In some embodiments, use by a non-professional is performed to generate a sequence of images to be further processed by a computer or by a mobile phone using its internal camera. A benefit of the invention is a reduction in surgical interventions to determine if a lesion is pathological and providing more relevant information which is not available with today's technology.

The recording may be analyzed by the patient, sent off site for analysis, analyzed by a health care professional, or by a designated software.

In some embodiments, a system for non-invasive diagnosis of neoplastic skin lesions is provided. The system comprises a compact illumination unit that provides illumination around a preselected skin area. The illumination is directly coupled to the skin surrounding an opaque orifice. The illumination propagates through the under skin layers by diffusion and illuminates the malignant area from under the skin. An imaging device positioned above the malignant area records the area twice; once with sub skin illumination and secondly with external illumination. The wavelength appropriate for penetrating under the skin to create the illumination effect and the camera probe is capable of detection and receives the light emitted by the skin surface under investigation to provide a recording of the data. Optionally, the system is further based on a mobile phone camera and comprises of an attachment to the mobile phone that includes optics and sub skin illumination. Optionally, the system further comprises an imaging unit that can capture and/or develop the image generated from the surface under investigation.

Additional software options provide mapping of lesions, its growth rate under the skin, and measurements of oxygen levels which may be archived for follow up and built-in tools to determine progression of growth.

The invention provides a rapid evaluation tool to quickly analyze multiple skin lesions in a short time period, as well as in a non-invasive manner, to assess which lesions express markers associated with cancer by analyzing images taken from above and under the skin.

### BRIEF DESCRIPTION OF THE DRAWINGS

FIG. 1 is an illustration of the 4 stages of Melanoma.

FIG. 2A shows a cross-section of one preferred embodiment featuring a cone-shaped device with two illumination devices and a camera.

FIG. 2B is an isometric representation of embodiment featuring a cone-shaped device.

FIG. 3A shows a cross-section of another embodiment, similar to the previous one with the exception of using a mobile device camera.

FIG. 3B is an isometric representation of using a mobile device.

FIG. 4A shows a cross-section of an embodiment in which there is only one laser and two cameras—one from above and one in an oblique angle to the skin surface.

FIG. 4B is an isometric representation of the embodiment in which there is one laser and two cameras.

FIG. 5A shows a cross-section of another embodiment in which the apparatus is cylinder-shaped and is comprised of two different parts—the base is stationary and the upper part rotates around its axis to provide a complete view on the tested skin area.

FIG. 5B shows an isometric view of the apparatus described in FIG. 5A.

FIG. 5C shows an example of using the apparatus described in FIG. 5A to achieve a tomographic view.

### DETAILED DESCRIPTION OF EMBODIMENTS

Although the following detailed description contains many specifics for the purpose of illustration, a person of ordinary skill in the art will appreciate that many variations and alterations to the following details are within the scope of the invention. Very narrow and specific examples are used to illustrate particular embodiments; however the invention described in the claims is not intended to be limited to only these examples, but rather includes the full scope of the attached claims. Accordingly, the following preferred embodiments of the invention are set forth without any loss of generality to, and without imposing limitations upon the claimed invention.

The following detailed description of the preferred embodiments is accompanied by drawings that form a part hereof, and in which specific embodiments are shown in an illustrative way. It is understood that other embodiments may be utilized and structural changes may be made without departing from the scope of the present invention.

For ease of explanation the present invention is described as embodiments of an apparatus and method for optically illuminating under skin formation suspected to be malignant, recording their image over a period of time and analyzing the behavior of images taken with under skin illumination from a variety of angles to provide an improved assessment tool for cancerous skin disorders. The device could be used as an over the counter tool or as a specialized diagnostic tool. Apparatus and methods are used for optical stimulation of other tissues, such as muscles and/or generating muscle action potentials. CMAP is one form of muscle action potential.

### To Summarize:

An apparatus comprising a diagnostic imaging device to be hand held, that will include an opaque tubular profile with an orifice adapted to be applied on a human skin portion. First light-emitting sources that are disposed around the orifice tube wall will illuminate towards the skin and outside the tubular profile, to emit radiation penetrating the skin and illuminating the enclosed skin portion by under the skin scattering. Second light emitting sources will illuminate the skin from above and inside the opaque tubular profile. A camera will be used for recording imaging of enclosed by tube skin portions, and a controller will activate the camera under different illuminations from above and under the skin.

Another method that could be used is with said apparatus, with added devices such as an adaptor mounted in front of a tablet or other mobile device for using its camera for imaging the said enclosed skin portion.

Furthermore, the apparatus could have a somewhat different configuration comprising a stationary mechanical element placed on the skin surface, having an interface plane on which the apparatus is mounted, and rotated around its axis to generate multiple views. The camera in this apparatus could also be mounted in an oblique direction to the examined skin portion.

The apparatuses described so far could also be used comprising multiple light-emitting sources having wavelength in the visible and IR operative to emit a specific wavelength visible or invisible to the human eye—according to controller input—and a computing element for storing and analyzing the changes as a function of time of suspected areas.

Alternatively, it is possible to use a camera capable to shift its focus to under the skin areas.

Another method that could be used is done by obtaining images from sub skin illumination of suspected malignant areas combined with images from above skin, analyzing the images and presenting the results by software. In this method, there would be a hand held diagnostic device, which will include an opaque tubular profile with an orifice adapted to be applied on a human skin portion, light-emitting sources that are disposed around the orifice tube wall, illuminating towards the skin and outside the tubular profile to emit radiation penetrating the skin and illuminating the enclosed skin portion by under the skin scattering, as well as second light emitting sources illuminating the skin from above and inside the opaque tubular profile. It'll also include a camera for recording imaging of enclosed by tube skin portion, and a controller for activating the camera under different illuminations under the skin and from above.

Instead, the said method could further comprise an adaptor mounted in front of a tablet or other mobile device for using its camera for imaging the said enclosed skin portion, as well as a mechanical element stationarily placed onto the skin surface and on its opposite side the apparatus is mounted and rotated around the skin surface perimeter. The camera could also be mounted in an oblique direction to the examined skin portion and generate images from different directions.

In addition, it is preferable to add multiple light-emitting sources having wavelength in the visible and IR operative to emit a specific wavelength visible or invisible to human eye, according to controller input, as well as a computing element for storing and analyzing the changes as a function of time of suspected areas.

Moreover, it is possible to make use of a camera capable to shift its focus to under the skin areas and take multiple pictures at various depths.

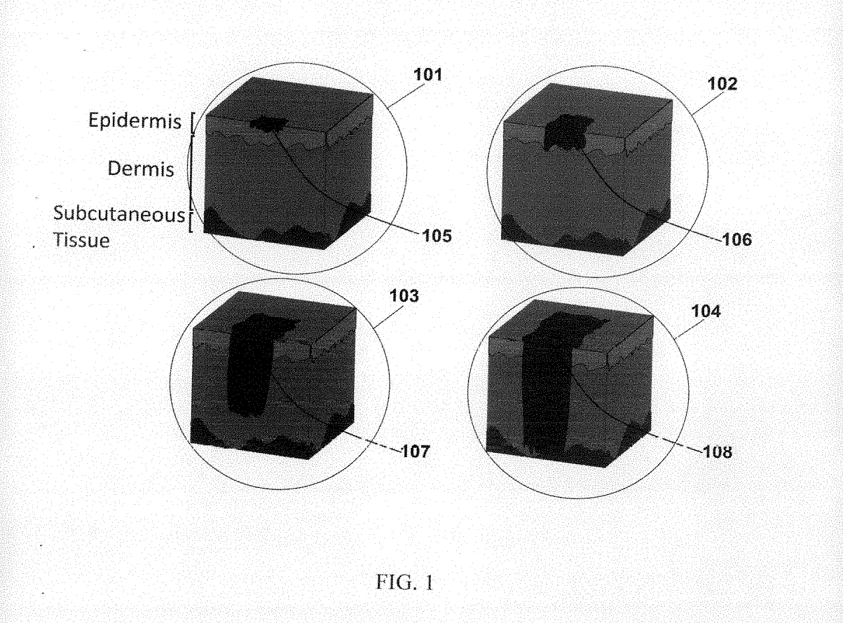

FIG. 1 illustrates the different stages of melanoma. The importance of sub skin melanoma progress is of outmost importance, both for diagnosis and treatment. There are several basic stages of melanoma; each one is given by different factors, while the most important one is the thickness of the tumor. On stage 1 denoted as 101 the tumor denoted as 105 is less than one millimeter thick, and if it is being detected at this stage, in most cases it can be cured with surgery. On stage 2 denoted as 102 the tumor denoted as 106 is above 1 millimeter in thickness, which means that in some cases it starts to be present at the Dermis layers. Stage 3 denoted as 103 is when the tumor denoted as 107 is between 2-4 millimeters in thickness, while stage 4 denoted as 104 means that the tumor denoted as 108 is thicker than 4 millimeters and has reached the subcutaneous tissue. For diagnostic purposes it is now clear why information revealing the tumor's depth and its under-skin behavior is very important, and potentially completes and improves available data preventing excessive and futile surgeries.

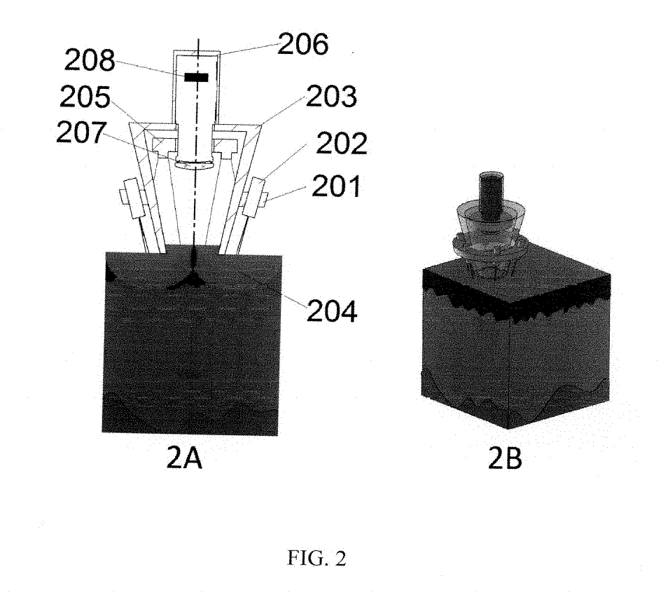

FIG. 2A is a preferred embodiment presented as a cross section of the 3-d picture denoted as 2B. This preferred embodiment is equipped with two illumination devices: 201 is based on a ring equipped with several light projectors denoted as 202, projecting multiple beams around an enclosed area where the suspected malignant skin is located. The enclosure denoted as 203 is opaque. The illumination penetrates through the skin and creates a lobe denoted as 204 which illuminates the skin from underneath, allowing better observation of features which otherwise would be difficult to see using illumination from above. The system is also equipped with yet another illumination ring denoted as 205 which illuminates the skin from above to allow recording with both types of illumination. A camera denoted as 206 is equipped with a lens 207 and an imaging sensor 208. By choosing illumination type, sub-skin and above-skin pictures are taken and later on are analyzed. Said lens 207 is free to move along its optical axis to allow focusing on various skin layers according to user activation.

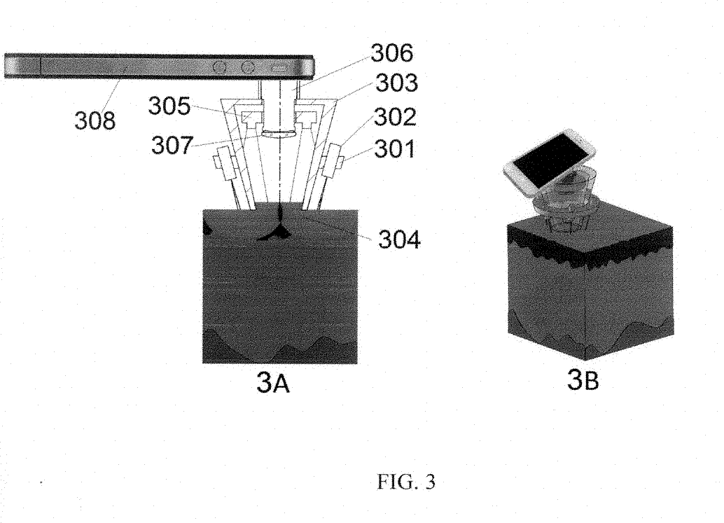

FIG. 3A is yet another preferred embodiment, is a cross section of the 3-d picture denoted as 3B. Here, a mobile device 308 has a lens attachment 306 having a built-in lens 307 for macro images of a skin portion denoted as 304. The selected skin portion 304 is enclosed by a tubular element 303 which is opaque to light. Multiple light sources disposed around the opaque tubular element illuminate the skin outside its perimeter. The light elements are denoted as 302 and their adaptor to the tubular element is denoted as 301. The light penetrates by scattering through the skin and illuminates the tumor from underneath. This increases the image quality and reveals otherwise obscured features either by skin thickness or by excessive illumination on skin top. For increasing the amount of gathered information the system is also equipped with above-the-skin illumination denoted as 305, so multiple images obtained from sub-skin or above-skin illumination or a combination of the two. The lens 307 can have a focusing feature that will focus onto the skin or beneath skin for further examination.

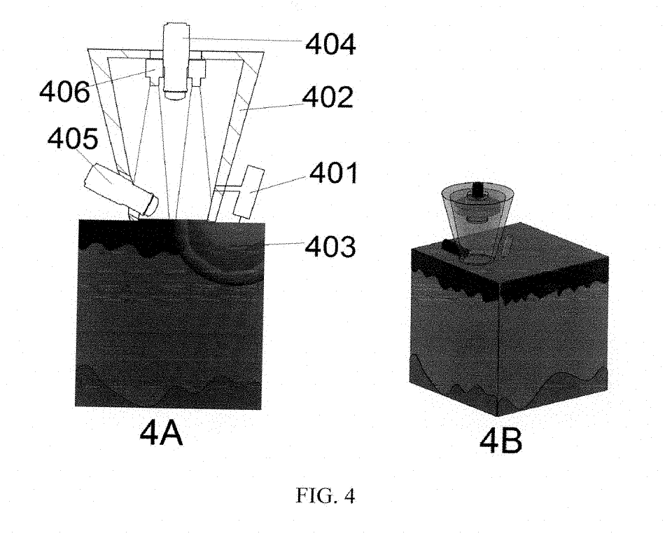

FIG. 4A discloses another preferred embodiment, with a cross section of the 3-d picture denoted as 4B. In this configuration, the outside illumination scheme 401 and the inside illumination 406 are identical to previous embodiments, same as members 402, 403 and 404. A new miniature camera 405 is obliquely positioned in respect with the optical axis of 404 element, to provide a better image of the under-skin melanoma progress. Multiple cameras could be displayed in a similar way around the tubular opaque element.

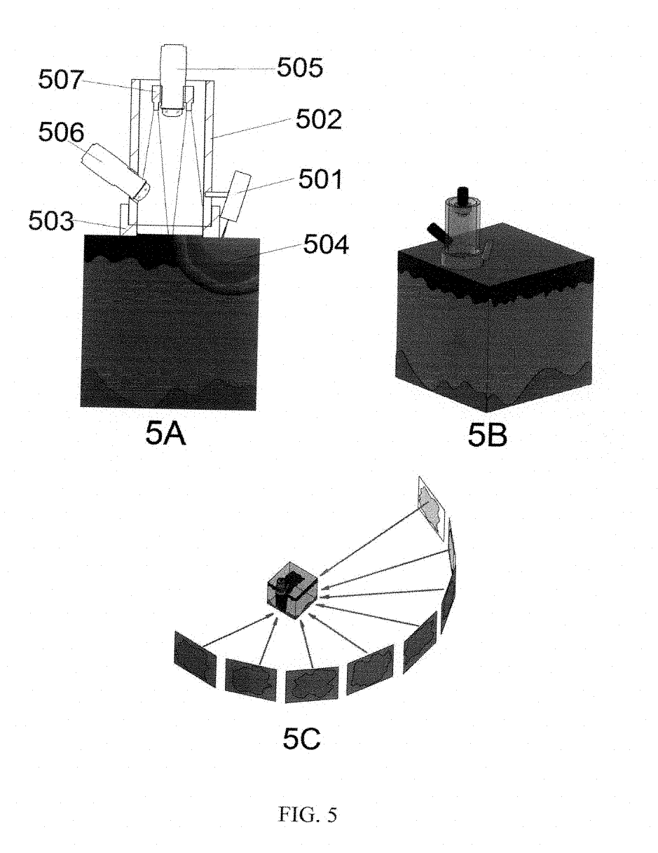

FIG. 5A is yet another disclosure of a preferred embodiment based on FIG. 4A concept. In this case multiple images around the suspected area are generated by rotating the imaging device of FIG. 4A around the preferred skin area. This layout has 3 explanatory views denoted as 5A, 5B and 5C. In this arrangement a stationary element 503 placed on the skin allows the disclosed configuration as per FIG. 4A to be rotated around its optical axis to generate multiple images using the oblique camera 506 from various angles. The generated images are displayed in a way of example in view 5C. Using tomographic reconstruction, the generated images could further be used to generate a 3-D model of the suspected skin area for further analysis and follow up. 501, 502, 504, 505, 507 have the same general functions as disclosed in FIG. 4A.

What is claimed is:

1. An apparatus comprising:
A diagnostic imaging device to be hand held, including:
An opaque tubular profile with an orifice adapted to be applied on a human skin portion.
First light-emitting sources that are disposed around the orifice tube wall, illuminating towards the skin and outside the tubular profile to emit radiation penetrating the skin and illuminating the enclosed skin portion by under the skin scattering.
Second light emitting sources illuminating the skin from above and inside the opaque tubular profile.
A camera for recording imaging of enclosed by tube skin portion.
-A controller for activating the camera under different illuminations under the skin and from above.

2. The apparatus of claim 1 further comprising an adaptor mounted in front of a tablet or other mobile device for using its camera for imaging of the said enclosed skin portion.

3. The apparatus of claim 1 further comprising of:
A stationary mechanical element placed on the skin surface, having an interface plane on which the apparatus is mounted, and rotated around its axis to generate multiple views.

4. The apparatus of claim 3 having its camera mounted in an oblique direction to the examined skin portion.

5. The apparatus of claims 1 to 4 further comprising:
Multiple light-emitting sources having wavelength in the visible and IR operative to emit a specific wavelength visible or invisible to the human eye, according to controller input.
A computing element for storing and analyzing the changes as a function of time of suspected areas.

6. The apparatus of claims 1 to 5 further comprising:
A camera capable to shift its focus to under the skin areas.

7. A method comprising:
Obtaining images from sub skin illumination of suspected malignant areas combined with images from above skin, analyzing the images and presenting the results by software.
A diagnostic imaging device to be hand held, including:
An opaque tubular profile with an orifice adapted to be applied on a human skin portion.
First light-emitting sources that are disposed around the orifice tube wall, illuminating towards the skin and outside the tubular profile to emit radiation penetrating the skin and illuminating the enclosed skin portion by under the skin scattering.
Second light emitting sources illuminating the skin from above and inside the opaque tubular profile.
A camera for recording imaging of enclosed by tube skin portion.
A controller for activating the camera under different illuminations under the skin and from above.

8. The method of claim 7 further comprising an adaptor mounted in front of a tablet or other mobile device for using its camera for imaging the said enclosed skin portion.

9. The method of claim 7 the further comprising of:
A stationary mechanical element placed on the skin surface, having an interface plane on which the apparatus is mounted, and rotated around its axis to generate multiple views.

10. The method of claim 9 having its camera mounted in an oblique direction to the examined skin portion to generate images from different directions.

11. The method of claims 7 to 10 further comprising:
Multiple light-emitting sources having wavelength in the visible and IR operative to emit a specific wavelength visible or invisible to human eye according to controller input.
A computing element for storing and analyzing the changes as a function of time of suspected areas.

12. The method of claims 7 to 11 further comprising:
A camera capable to shift its focus to under the skin areas and take multiple pictures at various depths.
